(19) United States
(12) Patent Application Publication
(10) Pub. No.: US 2024/0180436 A1
(43) Pub. Date: Jun. 6, 2024

(54) Systems, Devices and Methods for Noninvasively Determining Jugular Venous Pressure and/or Associated Metrics

(71) Applicant: Emory University, Atlanta, GA (US)

(72) Inventors: Lilas Dagher, Atlanta, GA (US); Camille Diaz Garcia, Atlanta, GA (US); Carlos Sabater, Atlanta, GA (US); Davis White, Atlanta, GA (US); Hannah Paterson, Atlanta, GA (US); Thomas Beckler, Atlanta, GA (US)

(21) Appl. No.: 18/531,138

(22) Filed: Dec. 6, 2023

Related U.S. Application Data
(60) Provisional application No. 63/386,261, filed on Dec. 6, 2022.

(51) Int. Cl. A61B 5/021; A61B 5/00; A61B 5/02; A61B 5/28

(52) U.S. Cl. CPC A61B 5/02125; A61B 5/02028; A61B 5/28; A61B 5/7221; A61B 5/7267; A61B 5/6833; A61B 5/742; A61B 2562/04

(57) ABSTRACT

The non-invasive devices and methods can accurately determine one or more measurements associated with jugular venous pressure while allowing for non-specific placement of (external) sensors. In some examples, the method may include obtaining a set of ECG data from one or more ECG sensors and a set of PPG data from two or more PPG sensors for a patient for a period of time. The method may further include segmenting the PPG data for each PPG sensor using the ECG data. The method may also include determining one or more measurements associated with jugular venous pressure using the segmented PPG data from the two or more PPG sensors.

### CROSS-REFERENCE TO RELATED APPLICATIONS

This application claims the benefit of U.S. Provisional Application No. 63/386,261 filed Dec. 6, 2022. The entirety of this application is hereby incorporated by reference for all purposes.

### BACKGROUND

Central venous pressure (CVP) measurement can be essential for monitoring hemodynamics in patients such as those individuals with heart failure. Current methods are performed within a hospital or clinical setting. The current standard technique for measurement of CVP is invasive procedure in which a catheter needs to be accurately inserted into a subclavian or internal jugular vein. This procedure can be costly and has potential complications.

CVP can be estimated non-invasively by physical examination of the jugular veins of the neck and estimation of jugular venous pressure (JVP). JVP measurements can be difficult and inaccurate due to variance in patient position and clinician measurement techniques. Thus, this method can generally not a reliable indicator of central venous pressure.

### SUMMARY

Thus, there is a need for an accurate, non-invasive measurement of jugular venous pressure that could be used to monitor and assess one or more hemodynamic metrics.

Techniques disclosed herein relate generally to non-invasive devices that can accurately determine one or more measurements associated with jugular venous pressure. The disclosed devices allow for non-specific external placement of the sensors. The devices need only to be placed externally in the approximate area of the internal jugular vein (IJV). The disclosed devices can therefore allow placement of the devices by less experienced healthcare professionals, as well as patients themselves for at-home monitoring.

The disclosed embodiments may include systems, devices, and methods for noninvasively measuring jugular venous pressure and/or associated metrics. In some embodiments, a method may include obtaining a set of electrocardiogram (ECG) data from one or more ECG sensors and a set of photoplethysmography (PPG) data from two or more PPG sensors for a patient for a period of time. The method may also include segmenting the PPG data for each PPG sensor using the ECG data. The method may further include determining one or more measurements associated with jugular venous pressure using the segmented PPG data from the two or more PPG sensors.

In some examples, the two or more PPG sensors may include a first PPG sensor and a second PPG sensor. In some examples, the determining the one or more measurements associated with the jugular venous pressure using the segmented PPG data may include determining pulse wave velocity using the segmented PPG data received from the first PPG sensor and the segmented PPG data from the second PPG sensor. In some examples, the one or more measurements associated with jugular venous pressure may be determined using the pulse wave velocity.

In some examples, the method may also include determining one or more hemodynamic metrics using the one or more measurements associated with the jugular venous pressure.

In some examples, the method may further include processing each PPG segment through a trained model to determine whether to classify each PPG segment as a jugular venous pulse (JVPE) signal. The method may also include comparing the JPVE signals to criteria to determine whether the JVPE signals are sufficient. The method may include causing PPG adjustment instructions to be displayed if the JVPE signals are insufficient. The method may also include determining the one or more measurements associated with the jugular venous pressure if the JVPE signals are sufficient.

In some examples, a system may include a set of PPG sensors configured to record PPG data for one or more periods of time. The system may also include a set of ECG sensors configured to record ECG data for one or more periods of time. The system may further include a controller to simultaneously record the PPG data and the ECG data for the one or more periods of time. The controller may be configured to determine the one or more measurements associated with the jugular pressure using the PPG data and the ECG data.

In some examples, the set of PPG sensors may be disposed on a patch. In some examples, the set of PPG sensors may include a plurality or sensors disposed in an array. In some examples, the set of PPG sensors may be configured to be separately and individually disposed on a target area.

In some examples, the system may also include one or more processors; and one or more hardware storage devices having stored thereon computer-executable instructions which are executable by the one or more processors to cause the computing system to perform at least the method described above.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the description. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1A illustrates an example of system environment for determining one or more measurements associated with jugular venous pressure and/or hemodynamic metrics according to embodiments.

FIG. 1B shows an enlarged view of the connection component shown in FIG. 1A.

FIG. 1C shows an enlarged view of a PPG sensor member shown in FIG. 1A.

FIGS. 2A-2G show examples of PPG sensor members according to embodiments.

FIG. 3 shows another example of a PPG sensor member according to embodiments.

FIG. 4 is a flow chart illustrating an example of a method of determining one or more measurements associated with jugular venous pressure and/or hemodynamic metrics according to embodiments.

FIG. 5 is an illustrative example of the use of ECG sensor data to segment PPG sensor data according to embodiments.

FIG. 6 is a simplified block diagram of an example of a computing system for implementing certain embodiments disclosed herein.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

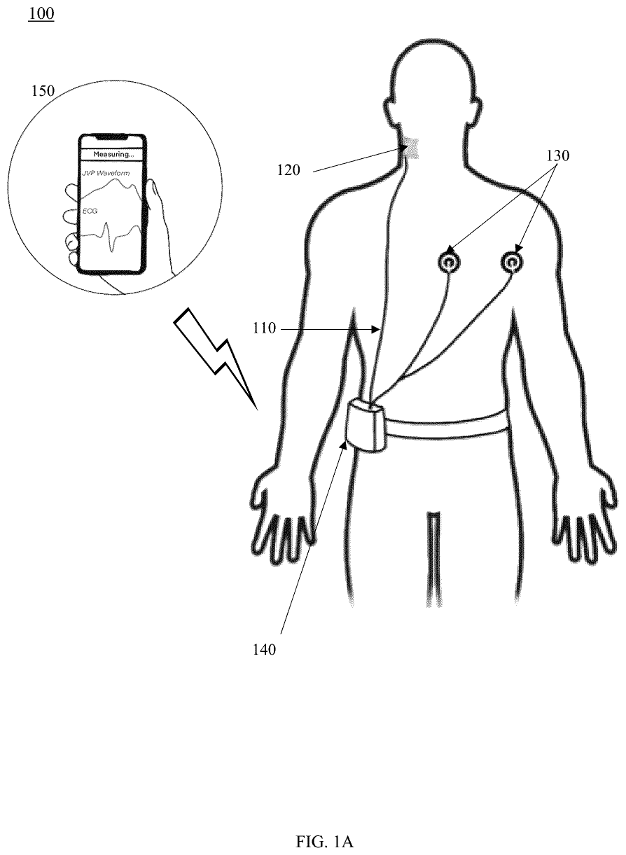

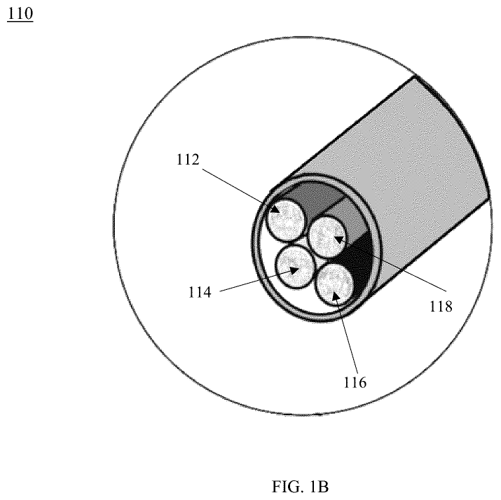

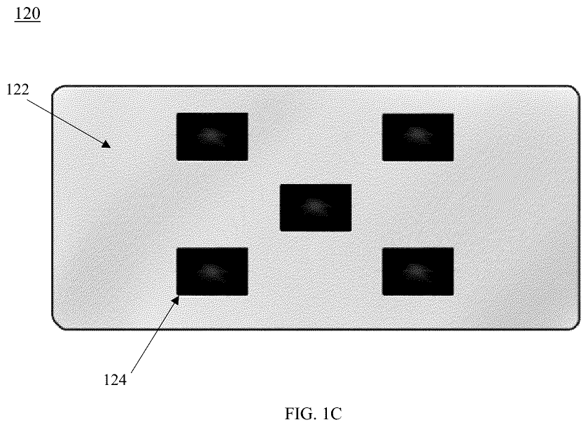

FIGS. 1A-C depict an example system environment 100 for noninvasively determining jugular venous pressure and/or one or more hemodynamic metrics according to embodiments. In some examples, the one or more hemodynamic metrics can include a qualitative and/or quantitative metric associated with jugular venous pressure measurement and/or heart failure. In some examples, the jugular venous pressure may be the measurement of jugular venous pressure in mm Hg, cm H₂O, other units of pressure, or any combination thereof.

The system 100 may include a device 102 that can be used to at least noninvasively acquire the sets of sensor data from two or more sets of sensors used to determine the jugular venous pressure and/or one or more hemodynamic metrics. In some examples, the two or more sets of sensors may include photoplethysmography (PPG) sensor(s), electrocardiogram (ECG) sensor(s), among others, or any combination thereof. In this example, the device 102 may include a set 120 of PPG sensors and a set 130 of ECG sensors.

In some examples, the device 102 may include a battery-powered controller 140 to control the acquisition and process the sensor data from the two or more sets of sensors 120, 130. In some examples, the controller 140 may be configured to wirelessly communicate with a user interface 150 to display the hemodynamic metric(s) and/or jugular venous pressure. In some examples, the controller 140 and/or the user interface 150 may be a single device.

In some examples, the controller 140 and/or the user interface 150 may include any computing or data processing device consistent with the disclosed embodiments. In some embodiments, the controller 140 and/or the user interface 150 may incorporate the functionalities associated with a personal computer, a laptop computer, a tablet computer, a notebook computer, a hand-held computer, a personal digital assistant, a portable navigation device, a mobile phone, an embedded device, a smartphone, and/or any additional or alternate computing device/system. The controller 140 and/or the user interface 150 may transmit and receive data across a communication network.

In some examples, as shown in FIG. 1A, the set 120 and the set 130 may be physically connected to the controller 140 using wired connection members. For example, the set 120 and the set 130 may be connected to the controller using wired connection members 110. In some examples, each wired member 110 may include a power cable 112, a serial clock line 114, a serial data line 116, and ground cable 118. In some examples, the set 120 and/or 130 may be connected to a controller 140 using other wired connection members, wireless connection (e.g., variety of local wireless communication techniques, such as RF communication according to the 802.11 or Bluetooth specification sets, infrared (IR) communication according to the IRDA specification set, or other standard or proprietary telemetry protocols), among others, or any combination thereof.

In some examples, the set 120 of PPG sensors may be configured to measure pulse wave velocity, using at least two spaced PPG sensors of the set 120, from which the jugular venous pressure can be determined. In this example, the set 120 of PPG sensors may be disposed on a flexible substrate 122, as shown in FIG. 1C. In some examples, the flexible substrate 122 may have a biocompatible adhesive disposed on one side (not shown) that can temporarily attach to skin of a user. In some examples, the set 120 may include two or more PPG sensors disposed in a pattern, for example, as shown in FIGS. 1C and/or 2A-3. As shown in FIG. 1A, a single wired connection member 110 may connect the set 120 via the substrate 122 to the controller 140.

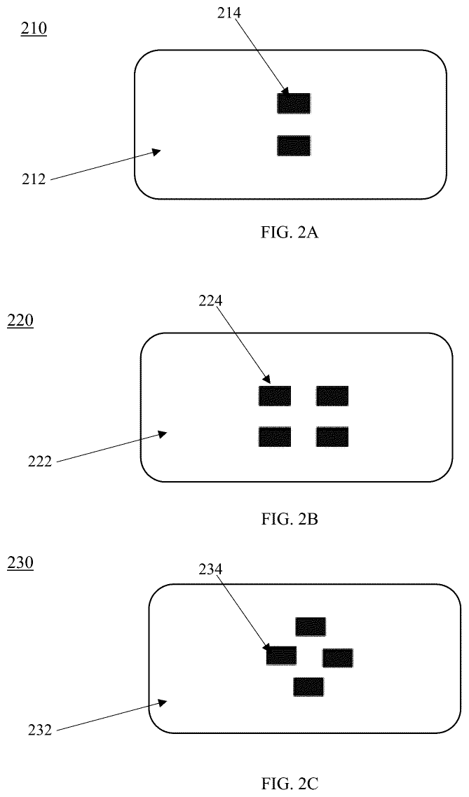

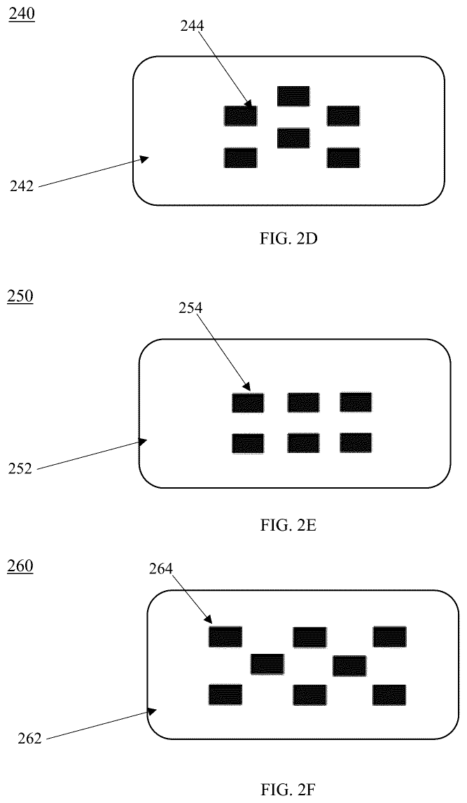

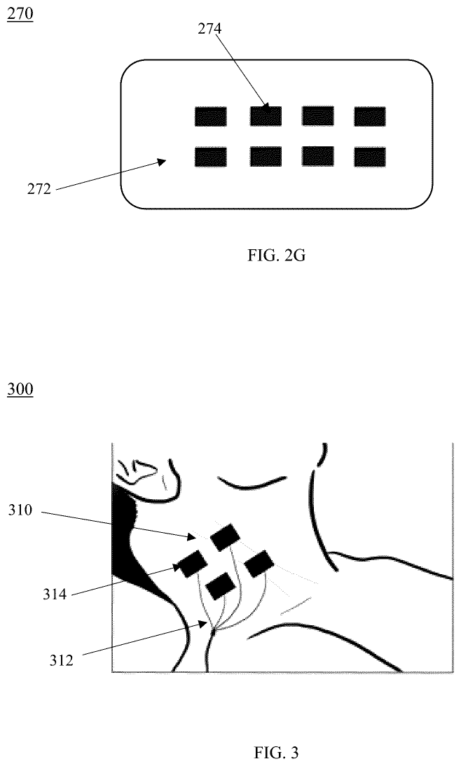

In the example shown in FIG. 1C, the set 120 may include five sensors in which two sensors are disposed on opposite sides of one sensor. In some examples, one or more different sensors, such as the ECG sensor, may also be disposed on the flexible substrate 122. In some examples, the set 120 may include a different number of PPG sensors, a different pattern of PPG sensors, a different configuration of PPG sensors, among others, or any combination thereof. FIGS. 2A-3 show different examples of the set of two or more PPG sensors.

In some examples, the set 130 of ECG sensors may include two or more ECG electrodes, for example, as shown in FIG. 1A. Each ECG electrode may be connected to the controller 140 using a wired connection member 110. In some examples, the set 130 may include additional or less ECG electrodes.

Although the systems/devices of the environment 100 are shown as being directly connected, the device 110 may be indirectly connected to one or more of the other systems/devices of the environment 100. In some embodiments, the device 110 may be only directly connected to one or more of the other systems/devices of the environment 100.

It is also to be understood that the environment 100 may omit any of the devices illustrated and/or may include additional systems and/or devices not shown. It is also to be understood that more than one device and/or system may be part of the environment 100 although one of each device and/or system is illustrated in the environment 100. It is further to be understood that each of the plurality of devices and/or systems may be different or may be the same. For example, one or more of the devices of the devices may be hosted at any of the other devices.

FIGS. 2A-2G show different examples of the set of PPG sensors disposed on a flexible substrate, like substrate 122, according to embodiments. It will be understood that these examples are nonlimiting and that the set of PPG sensors may include different number of sensors, pattern, configuration, among others, or any combination thereof.

FIG. 2A shows an example of a set 210 of two PPG sensors 214, spaced vertically, disposed on a flexible substrate 212. FIG. 2B shows an example of a set 220 of four PPG sensors 224, spaced evenly in a square shape, disposed on a flexible substrate 222. FIG. 2C shows an example of a set 230 of four PPG sensors 234, spaced evenly in a diamond shape, disposed on a flexible substrate 232. FIG. 2D shows an example of a set 240 of six PPG sensors 244, evenly spaced in columns and offset spaced in rows, disposed on a flexible substrate 242. FIG. 2E shows an example of a set 250 of six PPG sensors 254, spaced in evenly spaced rows and columns, disposed on a flexible substrate 252. FIG. 2F shows an example of a set 260 of eight sensors 264, evenly spaced in columns and alternately spaced in rows, disposed on a flexible substrate 262. FIG. 2G shows an example of a set 270 of eight sensors 274, evenly spaced in columns and rows, disposed on a flexible substrate 272.

In some examples, the set of PPG sensors may not be disposed on a single flexible patch. In some examples, each PPG sensor of the set of PPG sensors may be individually disposed, for example, on a patch, and individually connected to the controller 140 via its own connection member (e.g., wired connection member 110). FIG. 3 shows an example 300 of a set 310 of PPG sensors 314. In this example, each sensor 314 can be connected to a controller, such as the controller 140, using a wired connection member 312 (e.g., like wired connection member 110). In this example, the set 310 of PPG sensors may include four PPG sensors 314 as shown in FIG. 3. It will be understood that the set 310 is not limited to four PPG sensors 314 and may include more or less PPG sensors 314 (e.g., two sensors, three sensors, five sensors, six sensors, etc.).

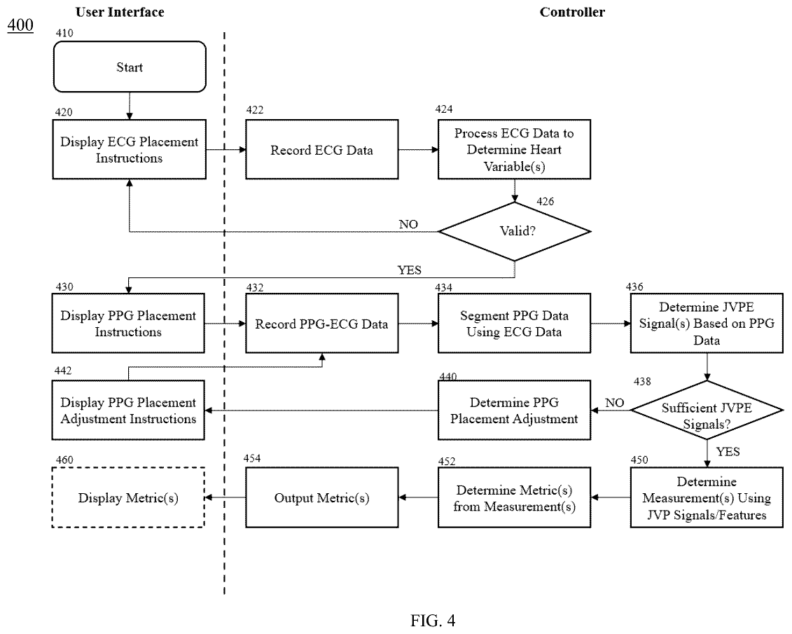

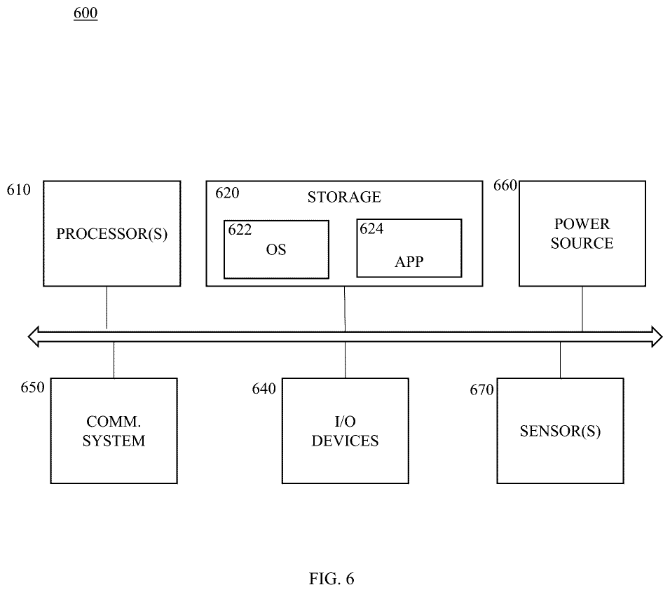

FIG. 4 shows a flow chart 400 illustrating an example of a method of determining jugular venous pressure and/or hemodynamic metrics according to certain embodiments. Operations described in the flow chart 400 may be performed by a computing system, such as the controller 140 and/or the user interface 150 described above with respect to FIG. 1A or a computing system described below with respect to FIG. 6. Although the flow chart 400 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in the flow chart 400 may begin at block 410, a sensor data collection session can be initiated at the controller 140 and/or user interface 150. For example, the user (e.g., patient and/or clinician) can initiate the session by pressing a button on the controller 140 and/or the user interface 150.

In some embodiments, once a session is initiated, at block 420, directions for placement of the set of ECG sensors, such as set 130, may be displayed at the user interface 150. In some examples, the controller 140 and/or the user interface 150 can confirm the proper placement of the set of ECG sensors before proceeding to step at block 422.

At block 422, in some examples, the controller 140 and/or the user interface 150 can record the ECG data from each sensor of the set for one or more periods of time. In some examples, the ECG data may be stored in a short-term memory.

At block 424, the controller 140 and/or the user interface 150 can process the ECG data to determine one or more cardiovascular variables (also referred to as “cardiovascular variables). In some examples, the one or more cardiovascular variables may include but is not limited to one or more cardiac pulse wave variables (e.g., R waves (also referred to as “peaks”)), heart rate variables (e.g., heart rate (also referred to as “pulse rate”), heart rate variability, among others, or any combination thereof), among others, or any combination thereof. In some examples, R peaks and heart rate may be determined using a peak finder algorithm.

In some examples, before determining the one or more cardiovascular variables, the ECG data may be preprocessed. For example, the ECG data may be filtered (e.g., using a Butterworth BPF filter), normalized, among others, or any combination thereof.

At block 426, the controller and/or the user interface can determine whether the ECG data is valid based on the one or more cardiovascular variables. For example, the ECG data would be determined to be valid if the ECG sensors are placed correctly. In some examples, the ECG sensors can be determined whether they are placed correctly based on whether the one or more cardiovascular variables are within a set range. For example, the heart rate may be compared to a set range; the dominant frequency, for example, determined using fast Fourier transform, to a set beats per minute range; the noise may be compared to a set noise range (e.g., using filters); among others; or a combination thereof.

If the ECG data is determined not to be valid (NO at block 426), the controller 140 and/or the user interface 150 may cause the steps at blocks 420-426 to be repeated until the controller 140 and/or the user interface 150 determines that the ECG data is valid.

If the ECG data is determined to be valid (YES at block 426), the controller 140 and/or the user interface 150 may cause the placement instructions of the set of PPG sensors (e.g., set 120) to be displayed at the user interface 150 at block 430. In some examples, the controller 140 and/or the user interface 150 can confirm the proper placement of the set of PPG sensors before proceeding to step at block 432.

Next, at block 432, the controller 140 and/or the user interface 150 can record the ECG data and PPG data from each sensor of the set for a period of time. In some examples, the ECG data and the PPG data may be stored in a short-term memory.

Next, at block 434, the controller 140 and/or the user interface 150 can segment the PPG data using the ECG data in some embodiments. In some examples, the PPG data may be segmented using the P-P interval determined from the ECG data.

In some examples, before segmentation, the PPG data and the ECG data can first be synchronized in time. In some examples, the PPG data and/or ECG data may be preprocessed to remove noise and/or outliers after synchronization but before segmentation. For example, the PPG data and/or the ECG data can be filtered (e.g., using Butterworth Bandpass filter), normalized, among others, or any combination thereof.

In some examples, after any synchronizing and/or preprocessing, the P-P interval can be determined from the ECG data. For example, the one or more cardiovascular variables may be determined from the ECG data. By way of example, the R peaks and the heart rate may be determined, for example, as discussed in the step at block 424. After which, the P-R wave interval may be determined from the heart rate, for example, using a regression analysis.

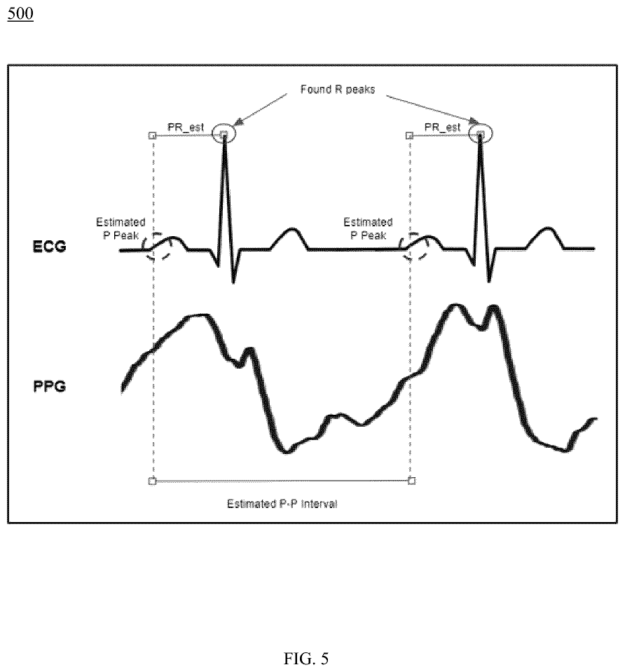

Using the P-R interval, the segmentation window may be determined in the ECG data. The window length may be equal to each R-R interval. After which, the P-P intervals may be determined for the ECG data. Using the P-P interval from the ECG data, a heart cycle may be segmented from the PPG data. FIG. 5 shows an example 500 of a visualization of the process of segmenting the PPG data using the estimated P-P interval determined from the ECG data.

In some examples, each PPG segment may be further processed to determine whether it is within a set range. If a PPG segment is determined to be outside the range, that segment can be removed from further analysis. By way of example, each PPG segment can be compared to a standard JVP template, an average value, among others, or any combination thereof.

Next, at block 436, the jugular venous pulse (JVPE) signals can be determined using each PPG segment. In some examples, the data in each PPG segment can be processed to determine whether it classifies as a JVPE. In some examples, the JVPE signal can be determined using a trained model, such as Random Forest Classifier, Support Vector Machines, extra tree classifier, k nearest neighbors, logistic regression, among others, or any combination thereof.

In some examples, the model may be a machine learning model trained using one or more features extracted from PPG data collected for a period of time at different measurement points (e.g., near the internal jugular, on the carotid artery, at locations with no discernable vessels, different locations on the neck, etc.) and corresponding ECG data for that period of time for a diverse group of users (e.g., different demographics, races, BMIs, different neck morphologies, ages, sexes, hemodynamic status (e.g., heart failure severity), etc.). For example, the one or more features may include but is not limited to waveform features, cardiac features, among others, or a combination thereof. In some examples, the waveform features may include but is not limited to number of 0 crossing, mean, median, mode, skewness, kurtosis, range, discrete wavelet transform coefficients, among others, or any combination thereof. In some examples, the cardiac features may include but is not limited to number of peaks, pulse area (area under PPG graph), pulse area after R peak from ECG, pulse area before R peak from ECG, highest value, lowest value, among others, or any combination thereof.

Before processing each PPG segment using a trained machine learning model, each PPG segment may be normalized. After which, one or more or more features may be determined for each normalized PPG segment and/or corresponding ECG segment. These features may be processed through the trained model to determine whether each PPG segment classifies as JVPE signal.

Next, at block 438, the controller 140 and/or the user interface 150 can determine whether there is a sufficient number of JVPE signals. In some examples, the controller 140 and/or the user interface 150 can determine whether a JVPE signal was received for two or more PPG sensors that meet set distance requirements.

If there is insufficient number of JVPE signals (NO at block 438), the controller 140 and/or the user interface 150 may determine PPG placement adjustment at block 440 and cause corresponding instructions to be displayed at the user interface at block 430. For example, the instructions may include moving one or more PPG sensors to a specific location, in a different direction, among others, or any combination thereof, so that at least two or more PPG sensors meet the set distance requirements. The steps at blocks 432-442 may be repeated until the controller 140 and/or the user interface 150 determines that there is a sufficient number of JVPE signals.

If there is a sufficient number of JVPE signals (YES at block 438), the controller 140 and/or the user interface 150 can determine one or more measurements associated with jugular venous pressure using the JVPE signals at block 450. In some examples, the one or more measurements associated with the jugular venous pressure may include but is not limited to pulse wave velocity.

In some examples, a time difference used to determine the pulse wave velocity can be determined between JVPE signals between one or more groups of two PPG sensors that meet the set distance requirements. In some examples, a time difference used to determine the pulse wave velocity can be determined using the first peak of the JVPE signal before the R wave from the corresponding ECG data, using cross-correlation between two signals, among others, or any combination thereof. After the time difference is determined, the pulse wave velocity may be determined by dividing the distance between each group of two PPG sensors by the corresponding time difference.

Next, in some examples, the controller 140 and/or the user interface 150 can determine one or more hemodynamic metrics using the one or more measurements associated with the jugular venous pressure at block 452. The one or more hemodynamic metrics may include one or more qualitative and/or quantitative metrics. For example, the one or more hemodynamic metrics may include a binary metric indicating an elevated jugular venous pressure as compared to the previous session for the user; a surrogate measurement for jugular venous pressure that indicates a hemodynamic status (e.g., lower jugular venous pressure); a pressure measurement (e.g., jugular venous pressure (JVP) in mmHg, cm H2O, or other pressure unit); among others, or any combination thereof. In some examples, JVP can be determined from the determined pulse wave velocity using a trained model.

Next, at block 454, the one or more metrics may be outputted. For example, the one or more metrics may be transmitted for display and/or storage to a healthcare information system, to the user interface 150, among others, or any combination thereof. In some examples, the one or more metrics may be displayed at the user interface 150 at block 460.

FIG. 6 depicts a block diagram of an example computing system 600 for implementing certain embodiments. For example, in some aspects, the computer system 600 may include computing systems associated with device(s) (e.g., the devices 140 and/or 150) performing one or more processes (e.g., FIG. 4) disclosed herein. The block diagram illustrates some electronic components or subsystems of the computing system. The computing system 600 depicted in FIG. 6 is merely an example and is not intended to unduly limit the scope of inventive embodiments recited in the disclosure. One of ordinary skill in the art would recognize many possible variations, alternatives, and modifications. For example, in some implementations, the computing system 600 may have more or fewer subsystems than those shown in FIG. 6, may combine two or more subsystems, or may have a different configuration or arrangement of subsystems.

In the example shown in FIG. 6, the computing system 600 may include one or more processing units 610 and storage 620. The processing units 610 may be configured to execute instructions for performing various operations, and can include, for example, a micro-controller, a general-purpose processor, or a microprocessor suitable for implementation within a portable electronic device, such as a Raspberry Pi®. The processing units 610 may be communicatively coupled with a plurality of components within the computing system 600. For example, the processing units 610 may communicate with other components across a bus. The bus may be any subsystem adapted to transfer data within the computing system 600. The bus may include a plurality of computer buses and additional circuitry to transfer data.

In some embodiments, the processing units 610 may be coupled to the storage 620. In some embodiments, the storage 620 may offer both short-term and long-term storage and may be divided into several units. The storage 620 may be volatile, such as static random access memory (SRAM) and/or dynamic random access memory (DRAM), and/or non-volatile, such as read-only memory (ROM), flash memory, and the like. Furthermore, the storage 620 may include removable storage devices, such as secure digital (SD) cards. The storage 620 may provide storage of computer readable instructions, data structures, program modules, audio recordings, image files, video recordings, and other data for the computing system 600. In some embodiments, the storage 620 may be distributed into different hardware modules. A set of instructions and/or code might be stored on the storage 620. The instructions might take the form of executable code that may be executable by the computing system 600, and/or might take the form of source and/or installable code, which, upon compilation and/or installation on the computing system 600 (e.g., using any of a variety of generally available compilers, installation programs, compression/decompression utilities, and the like), may take the form of executable code.

In some embodiments, the storage 620 may store a plurality of application modules 624, which may include any number of applications, such as applications for controlling input/output (I/O) devices 640 (e.g., a sensor (e.g., sensor(s) 670, other sensor(s), etc.)), a switch, a camera, a microphone or audio recorder, a speaker, a media player, a display device, etc.). The application modules 624 may include particular instructions to be executed by the processing units 610. In some embodiments, certain applications or parts of the application modules 624 may be executable by other hardware modules, such as a communication subsystem 650. In certain embodiments, the storage 620 may additionally include secure memory, which may include additional security controls to prevent copying or other unauthorized access to secure information.

In some embodiments, the storage 620 may include an operating system 622 loaded therein, such as an Android operating system or any other operating system suitable for mobile devices or portable devices. The operating system 622 may be operable to initiate the execution of the instructions provided by the application modules 624 and/or manage other hardware modules as well as interfaces with a communication subsystem 650 which may include one or more wireless or wired transceivers. The operating system 622 may be adapted to perform other operations across the components of the computing system 600 including threading, resource management, data storage control, and other similar functionality.

The communication subsystem 650 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. The computing system 600 may include one or more antennas (not shown in FIG. 6) for wireless communication as part of the communication subsystem 650 or as a separate component coupled to any portion of the system.

Depending on desired functionality, the communication subsystem 650 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, the communications subsystem 650 may include wired communication devices, such as Universal Serial Bus (USB) devices, Universal Asynchronous Receiver/Transmitter (UART) devices, Ethernet devices, and the like. The communications subsystem 650 may permit data to be exchanged with a network, other computing systems, and/or any other devices described herein. The communication subsystem 650 may include a means for transmitting or receiving data, such as identifiers of portable goal tracking devices, position data, a geographic map, a heat map, photos, or videos, using antennas and wireless links. The communication subsystem 650, the processing units 610, and the storage 620 may together comprise at least a part of one or more of a means for performing some functions disclosed herein.

The computing system 600 may include one or more I/O devices 640, such as sensors 670, a switch, a camera, a microphone or audio recorder, a communication port, or the like. For example, the I/O devices 640 may include one or more touch sensors or button sensors associated with the buttons. The touch sensors or button sensors may include, for example, a mechanical switch or a capacitive sensor that can sense the touching or pressing of a button.

In some embodiments, the I/O devices 640 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to the storage 620 or may be sent to the one or more network servers through the communication subsystem 650.

In some embodiments, the I/O devices 640 may include a location tracking device, such as a global positioning system (GPS) receiver. In some embodiments, the I/O devices 640 may include a wired communication port, such as a micro-USB, Lightning, or Thunderbolt transceiver.

The I/O devices 640 may also include, for example, a speaker, a media player, a display device, a communication port, or the like. For example, the I/O devices 640 may include a display device, such as an LED or LCD display and the corresponding driver circuit. The I/O devices 640 may include a text, audio, or video player that may display a text message, play an audio clip, or display a video clip.

The computing system 600 may include a power device 660, such as a rechargeable battery for providing electrical power to other circuits on the computing system 600. The rechargeable battery may include, for example, one or more alkaline batteries, lead-acid batteries, lithium-ion batteries, zinc-carbon batteries, and NiCd or NiMH batteries. The computing system 600 may also include a battery charger for charging the rechargeable battery. In some embodiments, the battery charger may include a wireless charging antenna that may support, for example, one of Qi, Power Matters Association (PMA), or Association for Wireless Power (A4WP) standard, and may operate at different frequencies. In some embodiments, the battery charger may include a hard-wired connector, such as, for example, a micro-USB or Lightning® connector, for charging the rechargeable battery using a hard-wired connection. The power device 660 may also include some power management integrated circuits, power regulators, power convertors, and the like.

In some embodiments, the computing system 600 may include one or more sensors 670. The sensors 670 may include, for example, the sensors as described above.

The computing system 600 may be implemented in many different ways. In some embodiments, the different components of the computing system 600 described above may be integrated to a same printed circuit board. In some embodiments, the different components of the computing system 600 described above may be placed in different physical locations and interconnected by, for example, electrical wires. The computing system 600 may be implemented in various physical forms and may have various external appearances. The components of computing system 600 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the disclosure.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as. For example, elements and/or features of different exemplary embodiments may be combined with each other and/or substituted for each other within the scope of this disclosure.

What is claimed:

1. A method for noninvasively determining one or more measurements associated with jugular venous pressure and/or associated metrics, comprising:
obtaining a set of electrocardiogram (ECG) data from one or more ECG sensors and a set of photoplethysmography (PPG) data from two or more PPG sensors for a patient for a period of time;
segmenting the PPG data for each PPG sensor using the ECG data; and
determining one or more measurements associated with jugular venous pressure using the segmented PPG data from the two or more PPG sensors.

2. The method according to claim 1, wherein the two or more PPG sensors include a first PPG sensor and a second PPG sensor, and wherein the determining the one or more measurements associated with the jugular venous pressure using the segmented PPG data includes:
determining pulse wave velocity using the segmented PPG data received from the first PPG sensor and the segmented PPG data from the second PPG sensor;
wherein the one or more measurements associated with the jugular venous pressure includes the pulse wave velocity.

3. The method according to claim 2, further comprising:
determining one or more hemodynamic metrics using the one or more measurements associated with the jugular venous pressure.

4. The method according to claim 3, further comprising:
outputting the one or more measurements associated with the jugular venous pressure and/or the one or more hemodynamic metrics.

5. The method according to claim 2, further comprising:
processing each PPG segment through a trained model to determine whether to classify each PPG segment as a jugular venous pulse (JVPE) signal;
comparing the JPVE signals to criteria to determine whether the JVPE signals are sufficient;
causing PPG adjustment instructions to be displayed if the JVPE signals are insufficient; and
determining the one or more measurements associated with the jugular venous pressure if the JVPE signals are sufficient.

6. A system, comprising:
a set of photoplethysmography (PPG) sensors configured to record PPG data for one or more periods of time;
a set of electrocardiogram (ECG) sensors configured to record ECG data for one or more periods of time; and
a controller to simultaneously record the PPG data and the ECG data for the one or more periods of time;
the controller being configured to determine one or more measurements associated with jugular pressure using the PPG data and the ECG data.

7. The system according to claim 6, wherein the set of PPG sensors are disposed on a patch.

8. The system according to claim 7, wherein the set of PPG sensors include a plurality or sensors disposed in an array.

9. The system according to claim 6, wherein the set of PPG sensors are configured to be separately disposed on a target area.

10. The system, further comprising:
one or more processors; and
one or more hardware storage devices having stored thereon computer-executable instructions which are executable by the one or more processors to cause the computing system to perform at least the following:
obtaining a set of electrocardiogram (ECG) data from one or more ECG sensors and a set of photoplethysmography (PPG) data from two or more PPG sensors for a patient for a period of time;
segmenting the PPG data for each PPG sensor using the ECG data; and
determining one or more measurements associated with the jugular venous pressure using the segmented PPG data from the two or more PPG sensors.

11. The system according to claim 10, wherein the two or more PPG sensors include a first PPG sensor and a second PPG sensor, and wherein the determining the one or more measurements associated with the jugular venous pressure using the segmented PPG data includes:
determining pulse wave velocity using the segmented PPG data received from the first PPG sensor and the segmented PPG data from the second PPG sensor;
wherein the one or more measurements associated with the jugular venous pressure includes the pulse wave velocity.

12. The system according to claim 11, wherein the one or more processors are further configured to cause the computing system to perform at least the following:
determining one or more hemodynamic metrics using the one or more measurements associated with the jugular venous pressure.

13. The system according to claim 12, wherein the one or more processors are further configured to cause the computing system to perform at least the following:
outputting the one or more measurements associated with the jugular venous pressure and/or the one or more hemodynamic metrics.

14. The system according to claim 11, wherein the one or more processors are further configured to cause the computing system to perform at least the following:
processing each PPG segment through a trained model to determine whether to classify each PPG segment as a jugular venous pulse (JVPE) signal;
comparing the JPVE signals to criteria to determine whether the JVPE signals are sufficient;
causing PPG adjustment instructions to be displayed if the JVPE signals are insufficient; and
determining the one or more measurements associated with the jugular venous pressure if the JVPE signals are sufficient.

15. The system according to claim 10, further comprising:
the two or more PPG sensors configured to record the PPG data for one or more periods of time; and
the one or more ECG sensors configured to record the ECG data for one or more periods of time.

16. The system according to claim 15, wherein the two or more PPG sensors are disposed on a patch.

17. The system according to claim 16, wherein the two or more PPG sensors include a plurality or sensors disposed in an array.

18. The system according to claim 15, wherein the two or more PPG sensors are configured to be separately disposed on a target area.
